(19) United States
(12) Patent Application Publication
(10) Pub. No.: US 2009/0155719 A1
(43) Pub. Date: Jun. 18, 2009

(54) Aromatic (meth)acrylate compound having an alpha-hydroxy, photosensitive polymer, resist compositions, and associated methods

(76) Inventors: Sang-Jun Choi, Seoul (KR); Youn-Jin Cho, Anyang-si (KR); Seung-Wook Shin, Uiwang-si (KR); Hye-Won Kim, Uiwang-si (KR)

Correspondence Address: LEE & MORSE, P.C., 3141 FAIRVIEW PARK DRIVE, SUITE 500, FALLS CHURCH, VA, 22042, US

(21) Appl. No.: 12/314,880

(22) Filed: Dec. 18, 2008

(30) Foreign Application Priority Data
Dec. 18, 2007 (KR) 10-2007-0133684

(51) Int. Cl. G03F 7/004; C07C 69/54; G03F 7/20; C08F 24/00

(52) U.S. Cl. 430/285.1; 560/221; 526/270; 430/322

(57) ABSTRACT

An aromatic (meth)acrylate compound having an α-hydroxy, the aromatic (meth)acrylate compound being represented by the following Formula 1:

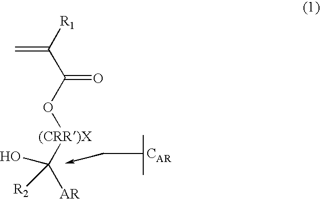

In Formula 1, R₁ is hydrogen or a methyl, R₂ is hydrogen, a substituted or unsubstituted alkyl, or a substituted or unsubstituted aryl, AR is a substituted or unsubstituted phenyl ring, or a substituted or unsubstituted aryl having from two to three fused aromatic rings, and carbon C_{AR} is bonded directly to an aromatic ring of AR, R and R′ are independently hydrogen or an alkyl, and X is an integer ranging from 1 to 6.

### BACKGROUND OF THE INVENTION

1. Field of the Invention

Embodiments relate to an aromatic (meth)acrylate compound having an α-hydroxy, a photosensitive polymer, a resist composition, and associated methods. More particularly, embodiments relate to an aromatic (meth)acrylate compound having excellent adhesion and dry etching resistance characteristics, a photosensitive polymer, a chemically amplified resist composition including the photosensitive polymer, and associated methods.

2. Description of the Related Art

For a photoresist material used to produce fine patterns, a deep-UV (deep UV) resist material using a shorter wavelength such that provided by an ArF excimer laser (193 nm) may be preferred to a resist material using a longer wavelength such as that provided by a KrF excimer laser (248 nm). For example, forming a semiconductor device with a capacity of more than 16 gigabytes needs a pattern size of less than 70 nm according to a design rule. As a result, a resist film may be thinner and have a reduced process margin for underlayer etching. The most representative problem is dry etching resistance of a photosensitive resin.

### SUMMARY OF THE INVENTION

Embodiments are therefore directed to an aromatic (meth)acrylate compound having an α-hydroxy, a photosensitive polymer, a resist composition, and associated methods, which substantially overcome one or more of the problems due to the limitations and disadvantages of the related art.

It is therefore a feature of an embodiment to provide an aromatic (meth)acrylate compound having an α-hydroxy, a photosensitive polymer, a resist composition, and associated methods, which may be particularly useful in photoresist applications for lithographic processes in the 193 nm wavelength region or shorter.

At least one of the above and other features and advantages may be realized by providing an aromatic (meth)acrylate compound having an α-hydroxy, the aromatic (meth)acrylate compound being represented by the following Formula 1:

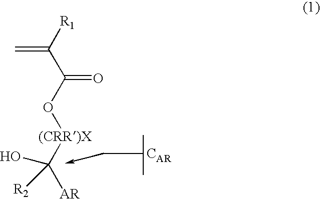

In Formula 1, R₁ may be hydrogen or a methyl, R₂ may be hydrogen, a substituted or unsubstituted alkyl, or a substituted or unsubstituted aryl, AR may be a substituted or unsubstituted phenyl ring, or a substituted or unsubstituted aryl having from two to three fused aromatic rings, carbon C_{AR} may be bonded directly to an aromatic ring of AR, R and R′ may independently be hydrogen or an alkyl, and X may be an integer ranging from 1 to 6.

AR may include first and second aromatic rings, the first and second aromatic rings being fused together, the first aromatic ring may have a group R₃ that may be hydrogen, a halogen, an alkyl, or an alkoxy, and the second aromatic ring may have a group R₄ that may be hydrogen, a halogen, an alkyl, or an alkoxy.

The aromatic (meth)acrylate compound may be represented by one of the following structures (a) to (k):

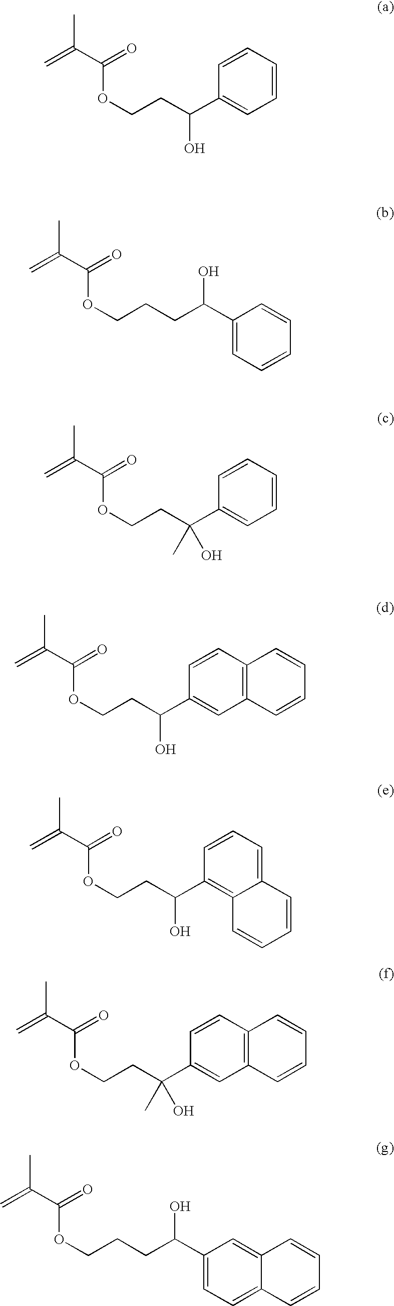

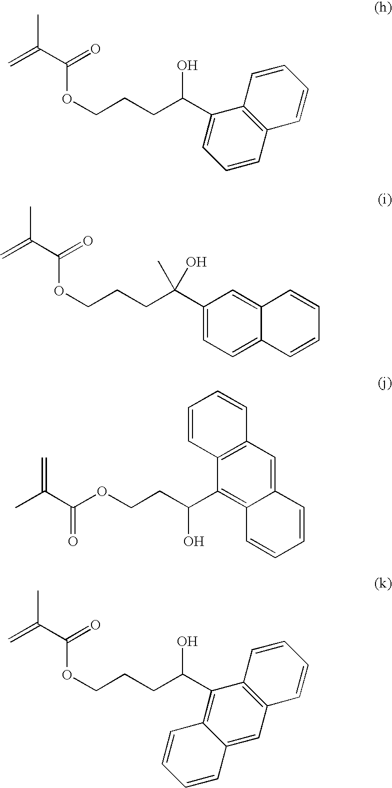

At least one of the above and other features and advantages may also be realized by providing a photosensitive aromatic (meth)acrylate polymer, the photosensitive aromatic (meth)acrylate polymer including repeating units represented by Formulae 6a, 6b, and 6c:

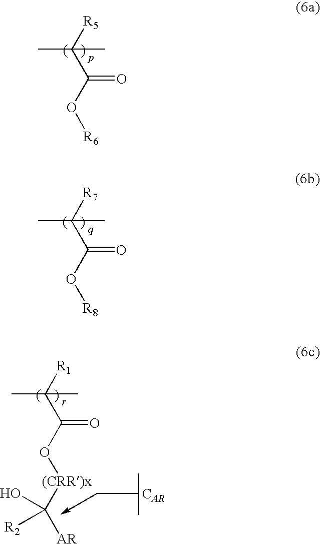

In Formulae 6a to 6c, R₁ may be hydrogen or a methyl, R₂ may be hydrogen, a substituted or unsubstituted alkyl, a substituted or unsubstituted aryl, or combinations thereof, AR may be a substituted or unsubstituted phenyl ring, or a substituted or unsubstituted aryl having from two to three fused aromatic rings, carbon C_{AR} may be bonded directly to an aromatic ring of AR, R and R′ may independently be hydrogen or an alkyl, R₅ and R₇ may independently be hydrogen or methyl, R₆ may be a C4 to C20 acid-labile group, R₈ may be a lactone-derived group, x may be an integer ranging from 1 to 6, p, q, and r are respectively mole ratios of the repeating units of Formulae 6a to 6c, p/(p+q+r) may be about 0.2 to about 0.5, q/(p+q+r) may be about 0.3 to about 0.5, and r/(p+q+r) may be about 0.1 to about 0.4.

R₆ may include one or more of norbornyl, isobornyl, cyclodecanyl, adamantyl, norbornyl having a lower alkyl substituent, isobornyl having a lower alkyl substituent, cyclodecanyl having a lower alkyl substituent, adamantyl having a lower alkyl substituent, alkoxycarbonyl, alkoxycarbonylalkyl, amyloxycarbonyl, amyloxycarbonylalkyl, 2-tetrahydropyranyloxycarbonylalkyl, 2-tetrahydrofuranyloxycarbonylalkyl, a tertiary alkyl, or an acetal.

R₈ maybe represented by at least one of Formula 4 or 5:

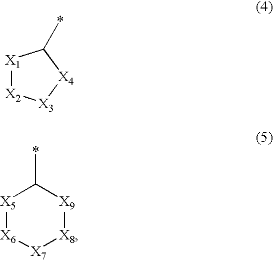

In Formula 4, at least two adjacent groups of X₁ to X₄ may independently be CO and O, and the remaining may be CR″ (where R″ is hydrogen, an alkyl, or an alkylene forming a fused ring with the five-member ring).

In Formula 5, at least two adjacent groups of X₅ to X₉ may independently be CO and O, the remaining may be CR″ (where R″ is hydrogen, an alkyl, or an alkylene forming a fused ring with the six-member ring), or all of X₅ to X₉ may be CR′″ (where R′″ is hydrogen, an alkyl, or an ester-containing alkylene forming a fused ring with the six-member ring) and at least two R′″ may be linked to each other to form a lactone ring.

The polymer may have a weight average molecular weight of about 3000 to about 20,000.

The polymer may have polydispersity of about 1.5 to about 2.5.

At least one of the above and other features and advantages may also be realized by providing a resist composition, including a photosensitive aromatic (meth)acrylate polymer according to an embodiment, a photoacid generator, and an organic solvent.

The photosensitive aromatic (meth)acrylate polymer may be included in an amount of about 5 to about 15 parts by weight, based on 100 parts by weight of the resist composition.

The photoacid generator may be included in an amount of about 1 to about 15 parts by weight, based on 100 parts by weight of the photosensitive aromatic (meth)acrylate polymer.

The photoacid generator may include one or more of a triarylsulfonium salt, a diaryliodonium salt, or a sulfonate.

The resist composition may further include an organic base, and the organic base may be present in an amount of about 0.1 to about 1.0 part by weight, based on 100 parts by weight of the photosensitive aromatic (meth)acrylate polymer.

The organic base may include one or more of triethylamine, triisobutylamine, trioctylamine, triisodecylamine, or triethanolamine.

At least one of the above and other features and advantages may also be realized by providing a method of patterning a material, the method including forming a resist layer on the material, forming a resist pattern from the resist layer using a lithographic process, and patterning the material through the resist pattern. The resist layer may include a photosensitive aromatic (meth)acrylate polymer according to an embodiment.

The lithographic process used to form the pattern in the resist layer may use light having a wavelength of 193 nm or shorter.

### BRIEF DESCRIPTION OF THE DRAWINGS

The above and other features and advantages will become more apparent to those of ordinary skill in the art by describing in detail exemplary embodiments with reference to the attached drawings, in which:

FIG. 1 illustrates Formula 1;

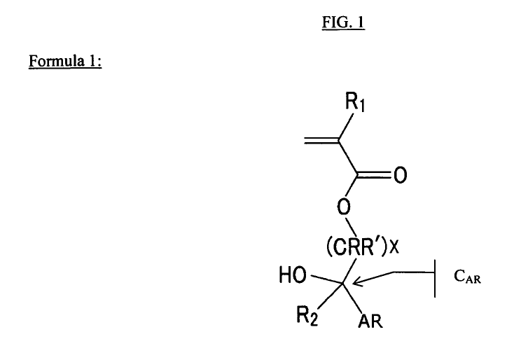

FIG. 2 illustrates Formulae (a)-(k);

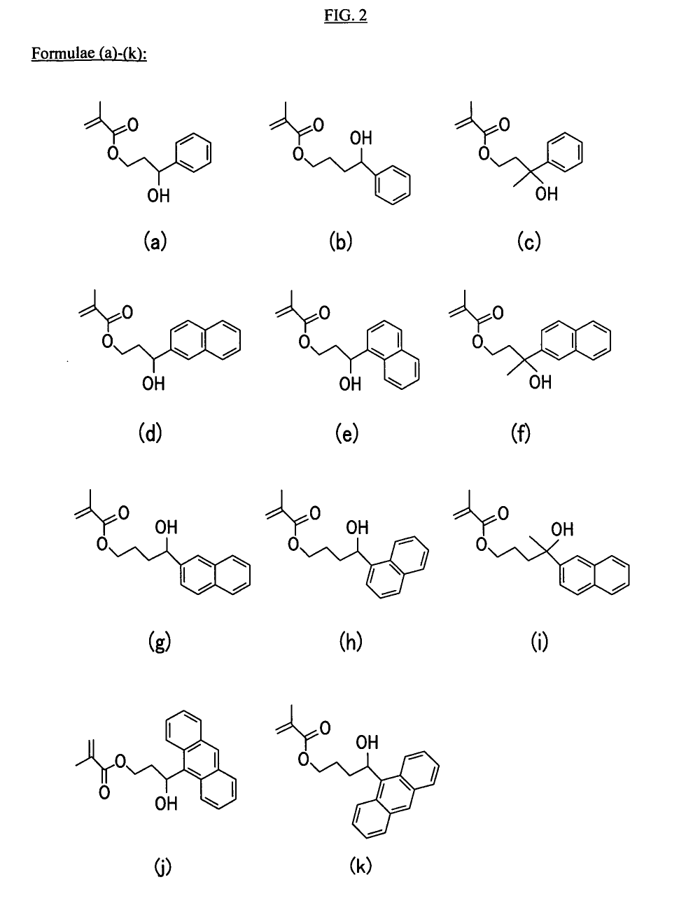

FIG. 3 illustrates Formulae 2-5;

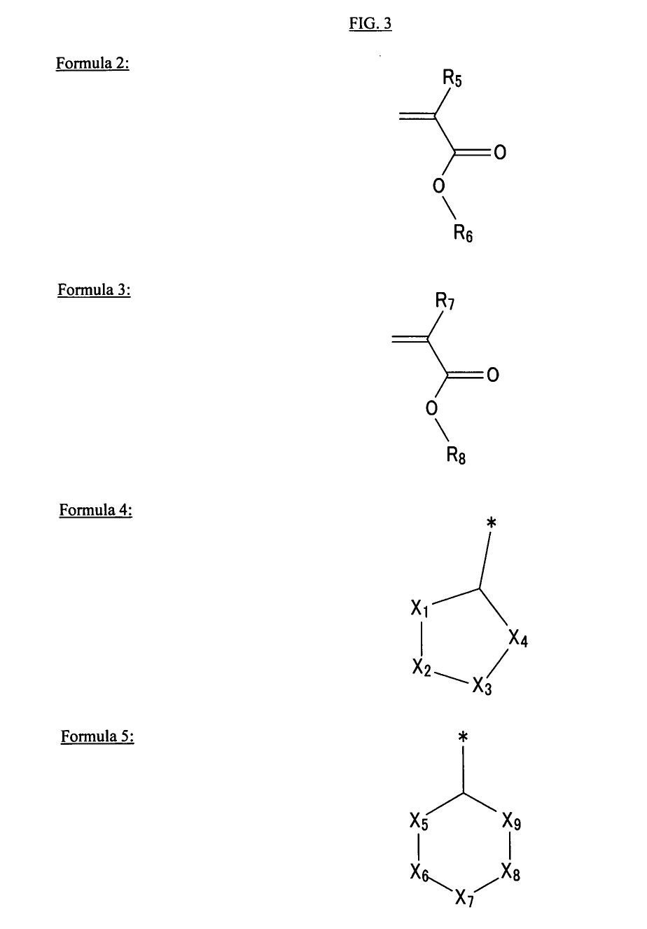

FIG. 4 illustrates Reaction Scheme 1;

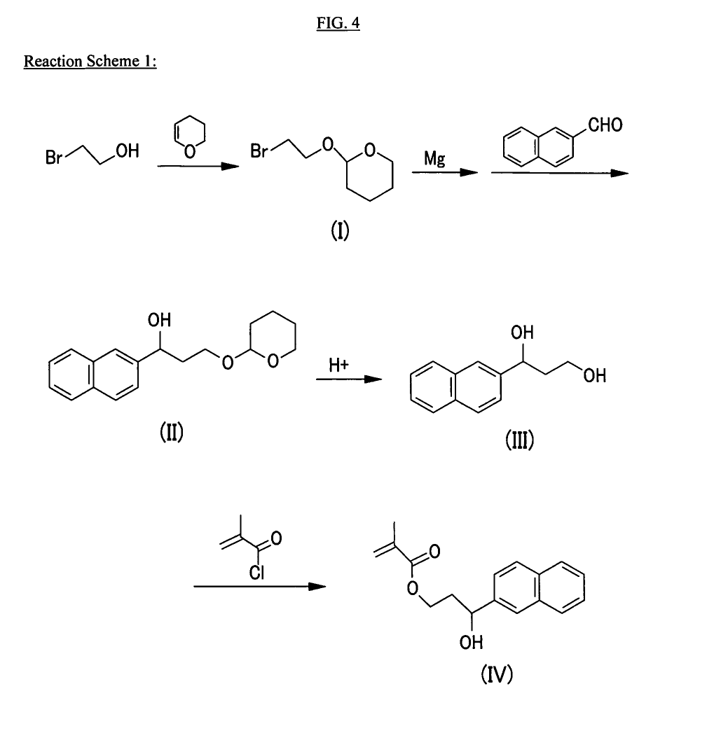

FIG. 5 illustrates Formulae 6a to 6c; and

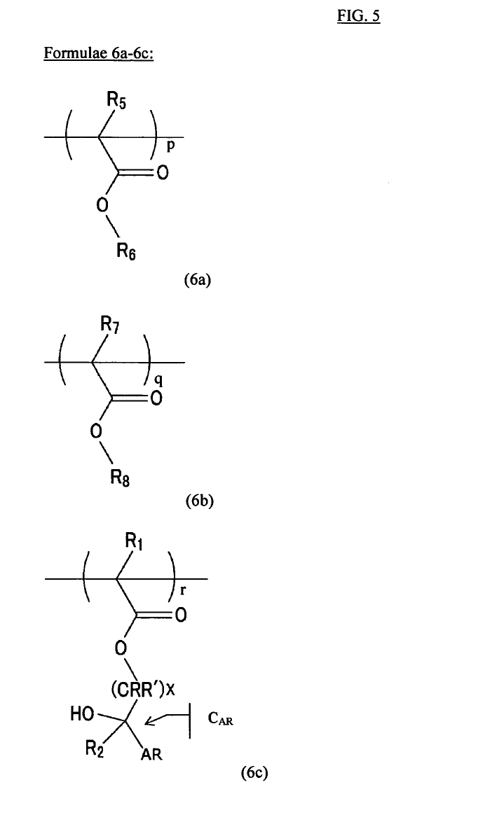

FIG. 6 illustrates Formulae 7-9.

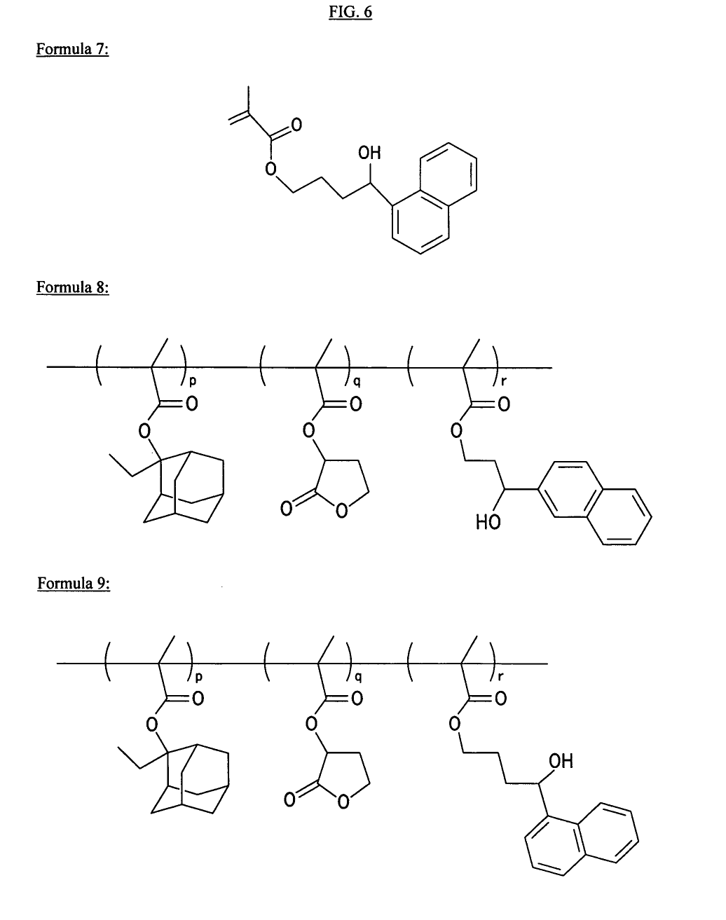

### DETAILED DESCRIPTION OF THE INVENTION

Korean Patent Application No. 10-2007-0133684, filed on Dec. 18, 2007, in the Korean Intellectual Property Office, and entitled: “Aromatic (Meth)Acrylate Compound, Photosensitive Polymer, and Resist Composition,” is incorporated by reference herein in its entirety.

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey the scope of the invention to those skilled in the art.

As used herein, the expressions “at least one,” “one or more,” and “and/or” are open-ended expressions that are both conjunctive and disjunctive in operation. For example, each of the expressions “at least one of A, B, and C,” “at least one of A, B, or C,” “one or more of A, B, and C,” “one or more of A, B, or C” and “A, B, and/or C” includes the following meanings: A alone; B alone; C alone; both A and B together; both A and C together; both B and C together; and all three of A, B, and C together. Further, these expressions are open-ended, unless expressly designated to the contrary by their combination with the term “consisting of.” For example, the expression “at least one of A, B, and C” may also include an n^{th} member, where n is greater than 3, whereas the expression “at least one selected from the group consisting of A, B, and C” does not.

As used herein, the expression “or” is not an “exclusive or” unless it is used in conjunction with the term “either.” For example, the expression “A, B, or C” includes A alone; B alone; C alone; both A and B together; both A and C together; both B and C together; and all three of A, B, and C together, whereas the expression “either A, B, or C” means one of A alone, B alone, and C alone, and does not mean any of both A and B together; both A and C together; both B and C together; and all three of A, B, and C together.

As used herein, the terms “a” and “an” are open terms that may be used in conjunction with singular items or with plural items. For example, the term “a photoacid generator” may represent a single compound, e.g., triarylsulfonium triflate, or multiple compounds in combination, e.g., triarylsulfonium triflate mixed with 2,6-dinitrobenzyl sulfonate.

As used herein, molecular weights of polymeric materials are weight average molecular weights, unless otherwise indicated.

As used herein, the term “(meth)acrylate” refers to both acrylate and methacrylate. Thus, for example, the term ethyl (meth)acrylate refers to both ethyl acrylate and ethyl methacrylate. Further, the term “acrylate” is generic to both acrylate and methacrylate, unless specified otherwise. Thus, ethyl acrylate and ethyl methacrylate are both acrylates.

As used herein, unless specified otherwise, the term “an alkyl” refers to a C1 to C20 alkyl and preferably a C1 to C12 alkyl, the term “a lower alkyl” refers to a C1 to C4 alkyl, the term “an alkoxy” refers to a C1 to C20 alkoxy and preferably a C1 to C1 2 alkoxy, the term “an alkylene” refers to a C1 to C20 alkylene and preferably a C1 to C12 alkylene, the term “an aryl” refers to a C6 to C20 aryl and preferably a C6 to C12 aryl, and the term “cycloalkyl” refers to a C3 to C14 cycloalkyl.

Embodiments provide an aromatic (meth)acrylate compound having an α-hydroxy, a photosensitive polymer, a resist composition, and associated methods According to an embodiment, an aromatic (meth)acrylate compound having an α-hydroxy may be represented by the following Formula 1.

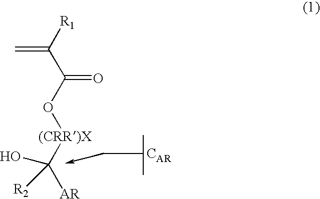

In Formula 1, R₁ may be hydrogen or a methyl, R₂ may be hydrogen, a substituted or unsubstituted alkyl, or a substituted or unsubstituted aryl, AR may be a substituted or unsubstituted phenyl ring, or a substituted or unsubstituted aryl having from two to three fused aromatic rings, carbon C_{AR} may be bonded directly to an aromatic ring of AR, R and R′ may independently be hydrogen or an alkyl, and X may be an integer ranging from 1 to 6.

The group AR of the Formula 1 may be, e.g., benzene, naphthalene, or anthracene. The group AR may include first and second aromatic rings, the first and second aromatic rings being fused together. The first aromatic ring may have a group R₃ that may be hydrogen, a halogen, an alkyl, or an alkoxy. The second aromatic ring may have a group R₄ that may be hydrogen, a halogen, an alkyl, or an alkoxy. R₃ and R₄ may be the same or different. In an implementation, the carbon C_{AR} may be bonded to the first aromatic ring having the group R₄.

Specific examples of the aromatic (meth)acrylate having an α-hydroxy represented by Formula 1 may include compounds represented by the following Formulae (a) to (k).

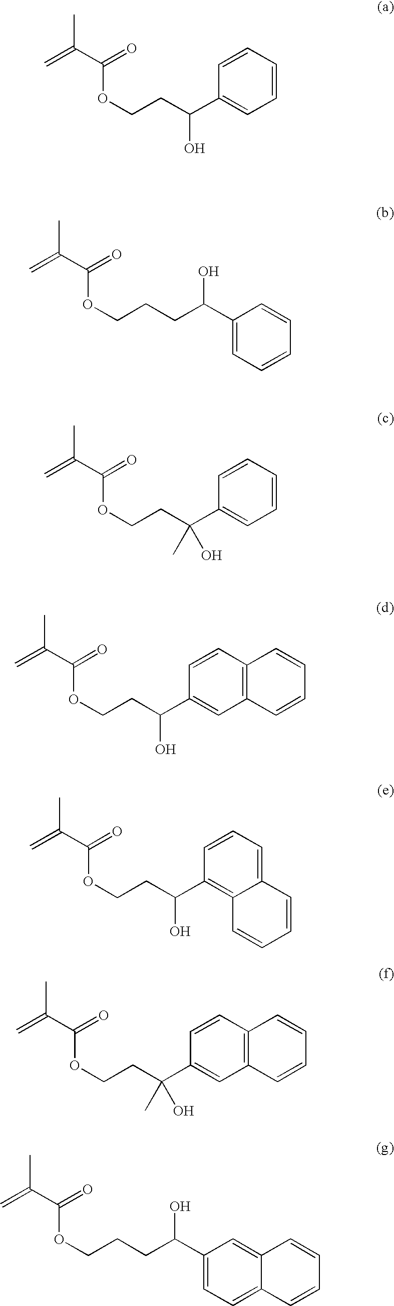

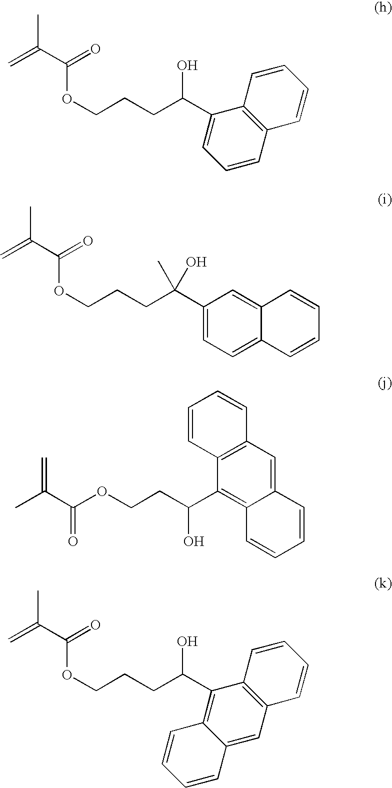

The aromatic (meth)acrylate compound having an α-hydroxy may be synthesized by reacting Grignard reactants of aromatic aldehyde or aromatic ketone compounds at various α- or β- with a (meth)acryloyl halide such as (meth)acryloyl chloride.

In particular, an aromatic (meth)acrylate compound of Formula 1 may be prepared by: preparing a primary reaction product by using an alcohol-protection specimen, e.g., dihydropyran, from an alcohol halide material, e.g., bromoethanol; forming the primary reaction product into a Grignard reactant by using magnesium (Mg); preparing a secondary reaction product by reacting the Grignard reactant with an aromatic aldehyde or aromatic ketone at α- or β-; and submitting the secondary reaction product to an alcohol-deprotection reaction and then reacting it with a (meth)acryloyl halide, e.g., acryloyl chloride and methacryloyl chloride, etc.

Since the aromatic(meth)acrylate compound may include a hydroxyl group for improving the adhesion characteristic to an underlayer at an a position in an aromatic substituent having dry etching resistance, it may have an excellent adhesion characteristic to an underlayer and excellent dry etching resistance in a lithographic process when it is used for a photosensitive polymer as a monomer. As a result, the resist material of an embodiment may overcome the problem of a conventional ArF resist material and can be satisfactorily used as an etching mask in a semiconductor device requiring high resolution.

According to another embodiment, a photosensitive polymer obtained from the aromatic (meth)acrylate having an α-hydroxy of the Formula 1 is provided.

The photosensitive polymer may further include repeating units derived from compounds of the following Formulae 2 and 3 as well as the aromatic (meth)acrylate having an α-hydroxy of an embodiment, e.g., Formula 1.

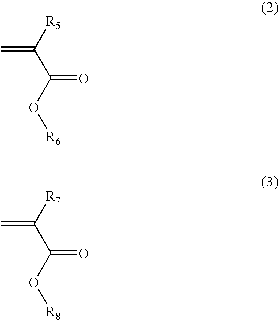

In Formulae 2 and 3, R₅ and R₇ may be the same or different and independently hydrogen or a methyl,

R₆ may be a C4 to C20 acid-labile group and may include, e.g., norbornyl, isobornyl, cyclodecanyl, adamantyl, norbornyl having a lower alkyl substituent, isobornyl having a lower alkyl substituent, cyclodecanyl having a lower alkyl substituent, adamantyl having a lower alkyl substituent, alkoxycarbonyl, alkoxycarbonylalkyl, amyloxycarbonyl, amyloxycarbonyl alkyl, 2-tetrahydropyranyloxycarbonylalkyl, 2-tetrahydrofuranyloxycarbonylalkyl, a tertiary alkyl, or an acetal, and in another embodiment, is 2-methyl-2-norbornyl, 2-ethyl-2-norbornyl, 2-methyl-2-isobornyl, 2-ethyl-2-isobornyl, 8-methyl-8-tricyclodecanyl, 8-ethyl-8-tricyclodecanyl, 2-methyl-2-adamantyl, 2-ethyl-2-adamantyl, 2-propyl-2-adamantyl, t-butoxycarbonyl, t-butoxycarbonylmethyl, t-amyloxycarbonyl, t-amyloxycarbonylmethyl, 1-ethoxyethoxycarbonylmethyl, 2-tetrahydropyranyloxycarbonylalkyl, 2-tetrahydrofuranyloxycarbonylalkyl, t-butyl, triethylcarbyl, 1-methyl cyclohexyl, 1-ethylcyclopentyl, t-amyl, or an acetal.

R₈ may be a lactone-derived group, and may include a substituent represented by Formula 4 or Formula 5 below.

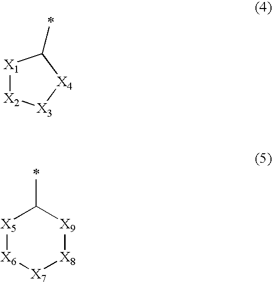

In Formula 4, at least two adjacent groups of X₁ to X₄ may independently be CO and O, and the remaining may be CR″ (where R″ is hydrogen, an alkyl, or an alkylene forming a fused ring with the five-member ring).

In Formula 5, at least two adjacent groups of X₅ to X₉ may independently be CO and O, and the remaining may be CR″ (where R″ is hydrogen, an alkyl, or an alkylene forming a fused ring with the six-member ring), or all of X₅ to X₉ may be CR′″ (where R′″ is hydrogen, an alkyl, or an ester-containing alkylene forming a fused ring with the six-member ring) and at least two R′″ are linked to each other to form a lactone ring.

In an embodiment, R₄ may be butyrolactonyl, valerolactonyl, 1,3-cyclohexanecarbolactonyl, 2,6-norbornanecarbolacton-5-yl, or 7-oxa-2,6-norbornanecarbolacton-5-yl.

The photosensitive polymer may be a random copolymer of aromatic (meth)acrylate having an α-hydroxy of Formula 1, and compounds of the Formulae 2 and 3. The photosensitive polymer may include repeating units represented by Formulae 6a, 6b, and 6c:

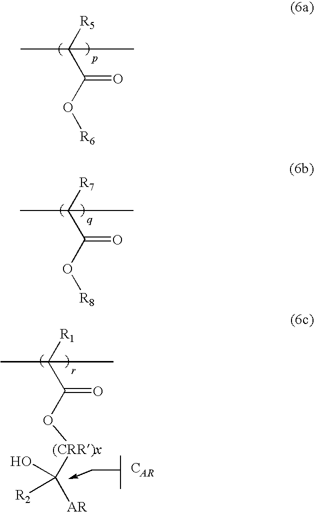

In Formulae 6a to 6c, R₁ to R₈, R, R′, AR, C_{AR}, and X may be the same as defined above.

In Formulae 6a to 6c, p, q, and r are mole ratios of the repeating units, p/(p+q+r) may be about 0.2 to about 0.5, q/(p+q+r) may be about 0.3 to about 0.5, and r/(p+q+r) may be about 0.1 to about 0.4.

The photosensitive polymer may have a weight average molecular weight (Mw) of about 3000 to about 20,000.

The photosensitive copolymer may have a polydispersity (ratio of weight average molecular weight to number average molecular weight, i.e., Mw/Mn) of about 1.5 to about 2.5, which may provide excellent etching resistance and resolution.

The photosensitive polymer according to an embodiment may be a copolymer obtained from new functional aromatic compounds, so it may have an advantage of providing a resist composition having both excellent adhesion to an underlayer and excellent dry etching resistance. When the resultant resist composition is applied in a photolithographic process, it may provide excellent lithography performance.

According to another embodiment, a resist composition including the photosensitive polymer is provided.

The resist composition may include the photosensitive polymer according to an embodiment, e.g., Formulae 6a to 6c, a photoacid generator (PAG), and a solvent.

Hereinafter, the components of the resist composition according to an embodiment are described in more detail.

Photosensitive polymer

The photosensitive polymer may be the same as described above, e.g., Formulae 6a to 6c derived from Formula 1 described above. The photosensitive polymer may be included in an amount of about 5 to about 15 parts by weight based on 100 parts by weight of the resist composition, which may provide the resist composition with excellent etching resistance and adhesion characteristics.

Photoacid Generator (PAG)

The photoacid generator may include an inorganic onium salt, organic sulfonate, or mixtures thereof. Specific examples of the photoacid generator include sulfonate or iodonium salt including a triarylsulfonium salt, a diaryl iodonium salt, sulfonate, or mixtures thereof. Preferable examples of the photoacid generator include triarylsulfonium triflate, diaryliodonium triflate, triarylsulfonium nonaflate, diaryliodonium nonaflate, succinimidyl triflate, 2,6-dinitrobenzyl sulfonate, or mixtures thereof.

The photoacid generator may be added at about 1 to about 15 parts by weight based on 100 parts by weight of the photosensitive polymer. Providing about 1 part by weight or more may help ensure that excessive exposure is not required. Providing about 15 parts by weight or less may help avoid decreases in the light transmission of the resist composition.

Solvent

The solvent may include, e.g., propylene glycol monomethyl ether acetate (PGMEA), propylene glycol methyl ether (PGME), ethyl lactate (EL), cyclohexanone, 2-heptanone, etc.

The solvent may be added as the balance amount of the composition. In an embodiment, the solvent may be added at about 80 wt % to about 95 wt %, based on 100 parts by weight of the entire resist composition.

Additive

The resist composition may further include an organic base (amine quencher) in order to control the exposure amount and to form a resist profile. The organic base may include, e.g., triethylamine, triisobutylamine, trioctylamine, triisodecylamine, triethanolamine, or mixtures thereof.

The amount of organic base may be present in the composition at about 0.1 to about 1 part by weight, based on 100 parts by weight of the photosensitive polymer. Providing about 0.1 parts by weight or more may help ensure a significant effect. Providing about 1 part by weight or less may help avoid undue increase in the amount of exposure required. Further, excessive organic base may impede pattern formation.

A process to form a desired pattern using the resist composition described above may be as follows.

A bare silicon wafer or a silicon wafer including a layer such as a silicon oxide layer, a silicon nitride layer, or a silicon nitride oxide layer on the upper surface thereof may be treated, e.g., with HMDS (hexamethyldisilazane) or an organic anti-reflection coating (bottom anti-reflective coating). Thereafter, the resist composition according to an embodiment may be coated on the silicon wafer at a thickness of, e.g., about 100 to about 150 nm.

The silicon wafer having the resist layer may be prebaked, e.g., at a temperature of about 90 to about 120° C. for about 60 to about 90 seconds, to remove solvent. A resist pattern may be formed from the resist layer using a process that includes, e.g., exposure to an exposure light source. The light source may be, e.g., ArF or a shorter wavelength, e.g., EUV (extreme UV), E-beam, and so on. In order to drive the chemical reaction in the exposed region of the resist layer, the layer may be subjected to PEB (post-exposure baking), e.g., at a temperature of about 90 to about 120° C. for about 60 to about 90 seconds.

Then the resist layer may be developed, e.g., in a basic aqueous developing solution such as a 2.38 wt % TMAH (tetramethylammonium hydroxide) solution. The exposure region may have a very high solubility in the basic aqueous developing solution, so it may be easily dissolved and removed during the development. When the exposure light source used is an ArF excimer laser, an 80 to 100 nm line and space pattern may be produced at an exposure dose of about 5 to about 50 mJ/cm².

The resist pattern obtained from the above lithographic process may be used as a mask, and the underlayer, e.g., an underlying silicon oxide layer, by using an etchant, e.g., a plasma of halogen gas or CₓF_{y} gas, such as a perfluorinated alkane in which x is a positive integer and y-2x+2. The resist pattern that remains on the wafer may be removed by using a stripper to yield a desired pattern in the target material layer, e.g., a silicon oxide layer pattern.

The following examples are provided in order to set forth particular details of one or more example embodiments. However, it will be understood that the embodiments described herein are not limited to the particular details described in the examples.

### EXAMPLE 1-1

### Synthesis of 3-hydroxy-3-(naphthalen-2-yl)propyl methacrylate monomer

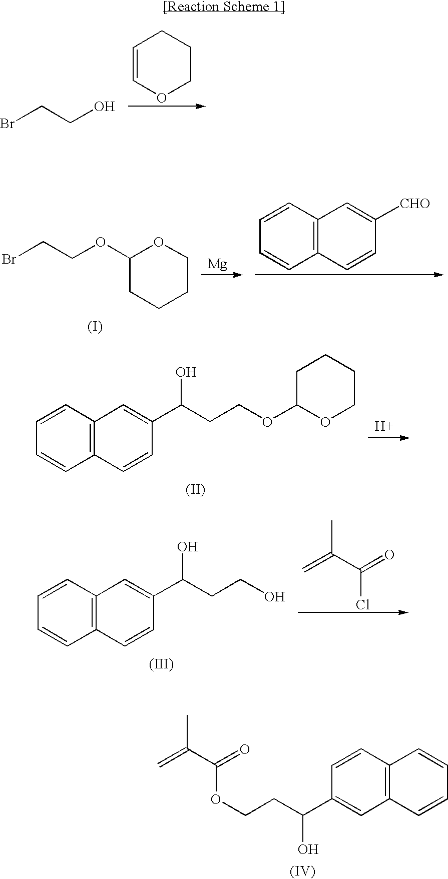

Using the method shown in Reaction Scheme 1, the 3-hydroxy-3-(naphthalen-2-yl)propyl methacrylate monomer (IV) was synthesized as follows. 15 g of 2-bromoethanol and 15 g of DHP (dihydropyran) was dissolved in 150 mL of methylene chloride, and p-toluene sulfonic acid (PTSA) was added thereto in a small amount. The resulting product was reacted at room temperature for about 4 hours. When the reaction was complete, the reactant was dripped into water to extract a primary reaction product (I) by using diethyl ether. Then, it was purified with vacuum distillation (yield: 90%).

Then, 3 g of Mg metal pieces was put in a round flask with a THF (tetrahydrofuran) solvent, and bromoethane in a catalyst amount was added thereto to activate the magnesium metal. 21 g of the primary reaction products (I) was slowly added to the mixture and then reacted at room temperature for two hours. Next, a 2-naphthaldehyde (15 g) solution was slowly dripped into the resulting reactant and then reacted at a temperature of about 45° C. for 8 hours. When the reaction was complete, the reactant was slowly neutralized with an excess amount of a diluted hydrochloric acid solution to extract a product (II) with diethyl ether and purified with column chromatography (hexane:ethyl acetate=3:1) (yield: 50%).

Then, 25 g of the acquired product (II) was dissolved in 200 mL of methylene chloride, and a hydrochloric acid (HCl) solution was added thereto in a small amount. The resulting mixture was reacted at room temperature for about 4 hours. The prepared product was dripped into an excess amount of water, extracted by using diethyl ether, and purified with column chromatography (hexane:ethyl acetate=2:1), acquiring a product (III) (yield: 80%).

20 g of the product (III) and 10 g of TEA (triethyl amine) were dissolved in 250 mL of THF, and 11 g of methacryloyl chloride was slowly dripped into the solution. The resulting mixture was reacted at room temperature for 4 hours. When the reaction was complete, the reactant was dripped into an excess amount of water. Then, the product was extracted by using diethyl ether and purified with column chromatography (hexane:ethyl acetate=2:1), acquiring a final product (IV) (yield: 50%).

¹H-NMR (CDCl₃, ppm): 7.8 (m, 3H, aromatic), 7.5 (m, 2H, aromatic),

7.2 (m, 2H, aromatic), 6.1 (s, 1H, vinyl), 5.6 (s, 1H, vinyl),

5.0 (m, 1H, —OH), 4.4 (m, 1H, —CH), 4.2 (m, 1H, —OCH₂₋),

2.2 (m, 2H, —CH₂₋), 1.9 (s, 3H, —CH₃)

FT-IR (NaCl, cm⁻¹): 3304 (—OH), 1736 (carbonyl ester)

### EXAMPLE 1-2

### Synthesis of 4-hvdroxy-4-(naphthalen-1-yl)butyl methacrylate monomer

A final product represented by Formula 7 was prepared according to the same method as Example 1 through reaction of a product with 1-naphthaldehyde after acquiring the product by using 3-bromo-1-propanol (yield: 50%).

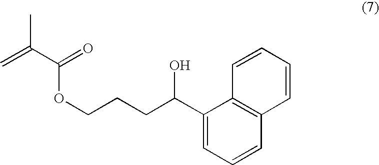

### EXAMPLE 2-1

### Synthesis of a photosensitive polymer

20 mmols of the 3-hydroxy-3-(naphthalen-2-yl)propyl methacrylate according to Example 1-1 was added to 40 mmols of y-butyrolactonyl methacrylate (GBLMA) and 40 mmols of 2-ethyl-2-adamantyl methacrylate (EAMA) in a round flask. They were dissolved with a dioxane solvent of a triple amount based on the total monomer weight therein, and 10 mmols of dimethyl-2,2′-azobis (2-methylpropionate) (V601, Wako Pure Chemical Industries Ltd.) was added thereto as a polymerization initiator. The mixture solution was polymerized at a temperature of 80° C. for 4 hours.

When the polymerization was complete, the reactant was slowly precipitated in an excess amount of a diethyl ether solvent. The precipitate was filtered, dissolved in an appropriate amount of THF, and reprecipitated in diethyl ether. Then, the precipitate was dried in a 50° C. vacuum oven for 24 hours, acquiring a polymer with the following Formula 8 (yield: 55%). Herein, the polymer had a weight average molecular weight (Mw) of 11,800 and polydispersity (Mw/Mn) of 1.8.

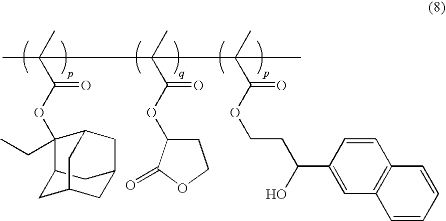

In Formula 8, p=4, q=4, and r=2.

### EXAMPLE 2-2

### Synthesis of a photosensitive polymer

20 mmols of the 4-hydroxy-4-(naphthalen-1-yl)butyl methacrylate according to Example 1-2 was added to 40 mmols of γ-butyrolactonyl methacrylate (GBLMA) and 40 mmols of 2-ethyl-2-adamantyl methacrylate (EAMA) in a round flask and then dissolved together in a dioxane solvent (monomer x 3 times), and 10 mmols of dimethyl-2,2′-azobis(2-methylpropionate) (V601, Wako Pure Chemical Industries Ltd.) was added thereto as a polymerization initiator. The mixture solution was polymerized at a temperature of 80° C. for 4 hours. When the polymerization was complete, a polymer of Formula 9 was acquired according to the same method as Example 2-1 (yield: 50%). Herein, the polymer had a weight average molecular weight (Mw) of 10,600 and polydispersity (Mw/Mn) of 1.8.

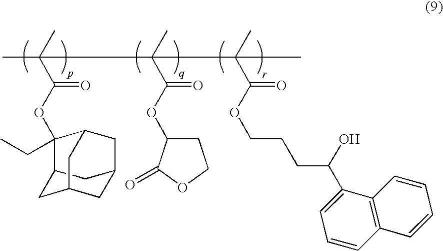

In Formula 9, p=4, q=4, and r=2.

### EXAMPLE 3

### Preparation of a resist composition and lithography performance

A resist composition was prepared by completely dissolving 0.8 g of a photosensitive polymer according to Example 2-1 and 0.02 g of TPS (triphenylsulfonium) nonaflate PAG in 17 g of PGMEA/EL (6/4), and then dissolving 1 mg of triethanolamine, an organic base, therein.

### EXPERIMENTAL EXAMPLE 1

### Resolution evaluation

The resist composition according to Example 3 was filtered by using a 0.1 μm thick membrane filter. The filtered resist composition was coated to be 140 nm thick on a silicon wafer treated with an organic BARC (AR_{46,} Rohm and Haas Company) to have a 600 Å thickness and soft-baked (SB) at a temperature of 110° C. for 60 seconds. It was exposed to light with an ArF scanner (0.78 NA, dipole), post-exposure baked (PEB), and then developed in a 2.38 wt % TMAH solution for 60 seconds.

As a result, a 90 nm L/S (line and space) pattern was acquired.

### EXPERIMENTAL EXAMPLE 2

### Etching resistance evaluation

The photosensitive polymer according to Example 2-1 was evaluated as to etching characteristics in a RIE (reactive ion etching) method by measuring bulk etching under CF₄ gas (composition: power of 100 W, pressure of 5P a, flow rate of 30 ml/min). The references were normalized with reference to an etching speed of a poly(hydroxystyrene) polymer, which is a resist for KrF.

As a result, it had a normalized etching speed of about 1.10 times that of a polymer for KrF.

Example embodiments have been disclosed herein, and although specific terms are employed, they are used and are to be interpreted in a generic and descriptive sense only and not for purpose of limitation. Accordingly, it will be understood by those of ordinary skill in the art that various changes in form and details may be made without departing from the spirit and scope of the present invention as set forth in the following claims.

What is claimed is:

1. An aromatic (meth)acrylate compound having an α-hydroxy, the aromatic (meth)acrylate compound being represented by the following Formula 1: 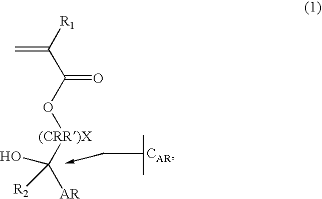
wherein, in Formula 1:
R₁ is hydrogen or a methyl,
R₂ is hydrogen, a substituted or unsubstituted alkyl, or a substituted or unsubstituted aryl,
AR is a substituted or unsubstituted phenyl ring, or a substituted or unsubstituted aryl having from two to three fused aromatic rings, and
carbon C_{AR} is bonded directly to an aromatic ring of AR,
R and R′ are independently hydrogen or an alkyl, and
X is an integer ranging from 1 to 6.

2. The aromatic (meth)acrylate compound as claimed in claim 1, wherein:
AR includes first and second aromatic rings, the first and second aromatic rings being fused together,
the first aromatic ring has a group R₃ that is hydrogen, a halogen, an alkyl, or an alkoxy, and
the second aromatic ring has a group R₄ that is hydrogen, a halogen, an alkyl, or an alkoxy.

3. The aromatic (meth)acrylate compound as claimed in claim 1, wherein the compound includes compounds having the following structures (a) to (k):

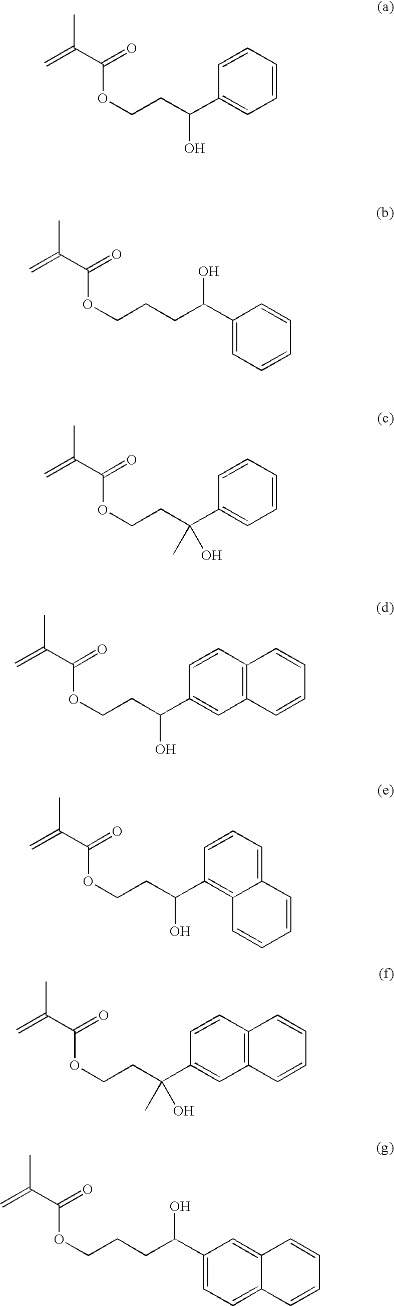

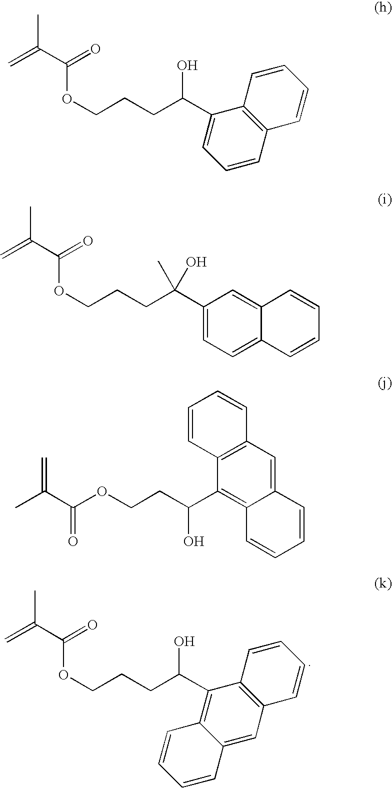

4. A photosensitive aromatic (meth)acrylate polymer, the photosensitive aromatic (meth)acrylate polymer including repeating units represented by Formulae 6a, 6b, and 6c: 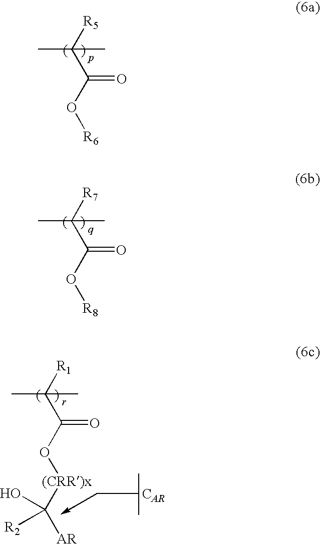
wherein, in Formulae 6a to 6c:
R₁ is hydrogen or a methyl,
R₂ is hydrogen, a substituted or unsubstituted alkyl, a substituted or unsubstituted aryl, or combinations thereof,
AR is a substituted or unsubstituted phenyl ring, or a substituted or unsubstituted aryl having from two to three fused aromatic rings,
carbon C_{AR} is bonded directly to an aromatic ring of AR,
R and R′ are independently hydrogen or an alkyl,
R₅ and R₇ are independently hydrogen or methyl,
R₆ is a C4 to C20 acid-labile group,
R₈ is a lactone-derived group,
x is an integer ranging from 1 to 6, and
p, q, and r are respectively mole ratios of the repeating units of Formulae 6a to 6c, p/(p+q+r) is about 0.2 to about 0.5, q/(p+q+r) is about 0.3 to about 0.5, and r/(p+q+r) is about 0.1 to about 0.4.

5. The polymer as claimed in claim 4, wherein R₆ includes one or more of norbornyl, isobornyl, cyclodecanyl, adamantyl, norbornyl having a lower alkyl substituent, isobornyl having a lower alkyl substituent, cyclodecanyl having a lower alkyl substituent, adamantyl having a lower alkyl substituent, alkoxycarbonyl, alkoxycarbonylalkyl, amyloxycarbonyl, amyloxycarbonylalkyl, 2-tetrahydropyranyloxycarbonylalkyl, 2-tetrahydrofuranyloxycarbonylalkyl, a tertiary alkyl, or an acetal.

6. The polymer as claimed in claim 4, wherein R₈ includes at least one of Formula 4 or 5: 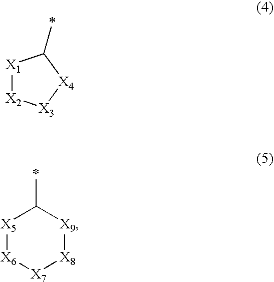
in Formula 4, at least two adjacent groups of X₁ to X₄ are independently CO and O, and the remaining are CR″ (where R″ is hydrogen, an alkyl, or an alkylene forming a fused ring with the five-member ring), and
in Formula 5:
at least two adjacent groups of X₅ to X₉ are independently CO and O, the remaining are CR″ (where R″ is hydrogen, an alkyl, or an alkylene forming a fused ring with the six-member ring), or
all of X₅ to X₉ are CR′″ (where R′″ is hydrogen, an alkyl, or an ester-containing alkylene forming a fused ring with the six-member ring) and at least two R′″ are linked to each other to form a lactone ring.

7. The polymer as claimed in claim 4, wherein the photosensitive polymer has a weight average molecular weight of about 3000 to about 20,000.

8. The polymer as claimed in claim 4, wherein the photosensitive polymer has polydispersity of about 1.5 to about 2.5.

9. A resist composition comprising:
a photosensitive aromatic (meth)acrylate polymer as claimed in claim 4;
a photoacid generator; and
an organic solvent.

10. The resist composition as claimed in claim 9, wherein the photosensitive aromatic (meth)acrylate polymer is included in an amount of about 5 to about 15 parts by weight, based on 100 parts by weight of the resist composition.

11. The resist composition as claimed in claim 9, wherein the photoacid generator is included in an amount of about 1 to about 15 parts by weight, based on 100 parts by weight of the photosensitive aromatic (meth)acrylate polymer.

12. The resist composition as claimed in claim 9, wherein the photoacid generator includes one or more of a triarylsulfonium salt, a diaryliodonium salt, or a sulfonate.

13. The resist composition as claimed in claim 9, further comprising an organic base, wherein the organic base is included in an amount of about 0.1 to about 1.0 part by weight of an organic base, based on 100 parts by weight of the photosensitive aromatic (meth)acrylate polymer.

14. The resist composition as claimed in claim 13, wherein the organic base includes one or more of triethylamine, triisobutylamine, trioctylamine, triisodecylamine, or triethanolamine.

15. A method of patterning a material, the method comprising:
forming a resist layer on the material;
forming a resist pattern from the resist layer using a lithographic process; and
patterning the material through the resist pattern, wherein:
the resist layer includes a photosensitive aromatic (meth)acrylate polymer, the (meth)acrylate polymer including repeating units represented by Formulae 6a, 6b, and 6c: 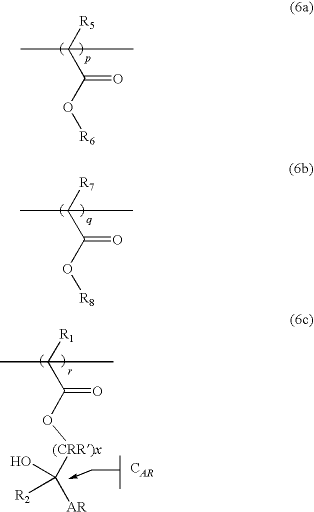
wherein, in Formulae 6a to 6c:
R₁ is hydrogen or a methyl,
R₂ is hydrogen, a substituted or unsubstituted alkyl, a substituted or unsubstituted aryl, or combinations thereof,
AR is a substituted or unsubstituted phenyl ring, or a substituted or unsubstituted aryl having from two to three fused aromatic rings,
carbon C_{AR} is bonded directly to an aromatic ring of AR,
R and R′ are independently hydrogen or an alkyl,
R₅ and R₇ are independently hydrogen or methyl,
R₆ is a C4 to C20 acid-labile group,
R₈ is a lactone-derived group,
x is an integer ranging from 1 to 6, and
p, q, and r are respectively mole ratios of the repeating units of Formulae 6a to 6c, p/(p+q+r) is about 0.2 to about 0.5, q/(p+q+r) is about 0.3 to about 0.5, and r/(p+q+r) is about 0.1 to about 0.4.

16. The method as claimed in claim 15, wherein the lithographic process used to form the pattern in the resist layer uses light having a wavelength of 193 nm or shorter.
